(19) United States
(12) Patent Application Publication
(10) Pub. No.: US 2022/0134000 A1
(43) Pub. Date: May 5, 2022

(54) Microdosing System, Apparatus, Method

(71) Applicant: Wave Neuroscience, Inc., Newport Beach, CA (US)

(72) Inventors: James William Phillips, Fountain Valley, CA (US); Alfred Jennings Walke, Encinitas, CA (US); Jonathan Ibasco, Long Beach, CA (US)

(21) Appl. No.: 17/317,731

(22) Filed: May 11, 2021

Related U.S. Application Data
(60) Provisional application No. 63/110,273, filed on Nov. 5, 2020.

(51) Int. Cl. A61M 5/172

(52) U.S. Cl. CPC A61M 5/1723; A61M 2230/10

(57) ABSTRACT

A microdosing delivery system and method is provided to improve dosage of a drug. Exemplary embodiments include system and methods for obtaining a one or more biological metrics in order to determine the dosage of the drug. Exemplary embodiments include system and method for delivering a dosage to a user based on the one or more biological metrics.

### PRIORITY

This application claims the benefit of priority under 35 U.S.C. § 119(e) to Provisional Application No. 63/110,273, filed on Nov. 5, 2020, which is incorporated by reference in its entirety.

### BACKGROUND OF THE INVENTION

The use of psychoactive drugs as a therapeutic has seen increased growth and popularity. For example, it has been shown that the practice of microdosing very low, sub-hallucinogenic doses of a psychedelic substance such as lysergic acid diethylamide (LSD) or psilocybin-containing mushrooms produces therapeutic and advantageous effects. Some of these advantages include, but are not limited to, improved mood, focus, self-efficacy, creativity, productivity, and social benefits.

Microdosing is generally based on the idea that the less you consume, the fewer drawbacks or side affects you are burdened with from the drug. With a controlled sub-perceptual dose, patients enjoy the maximum therapeutic benefits of the psychedelic without the general side effects associated with a greater dosage. However, effects depend on the drug used, user characteristics (such as gender, weight, or metabolism), personal tolerance, and other factors. Thus, correct individualized patient dosage amount and delivery are vital in optimizing the therapeutic outcome while reducing unwanted side effects.

As such, there is a need for determining an optimal microdosing amount based on biological metrics and outcome measures.

### SUMMARY OF THE INVENTION

The present disclosure relates to microdosing technology. Specifically, to electroencephalogram (EEG) controlled microdosing delivery.

The various embodiments of the present microdosing system, apparatus, method and computer-readable medium, have several objectives or benefits, in which no single one is essential to the microdosing system, apparatus, method, described herein. Accordingly, any component, feature, step or part may be integrated, separated, sub-divided, removed, duplicated, added, or used in any combination and remaining within the scope of the present disclosure. Embodiments are exemplary only, and provide an illustrative combination of features, but are not limited thereto.

### BRIEF DESCRIPTION OF THE DRAWINGS

FIG. 1 depicts a microdosing system according to an exemplary embodiment of the present disclosure.

FIG. 2 is a flowchart showing operation of the microdosing system according to an embodiment of the present disclosure.

### DETAILED DESCRIPTION OF THE EMBODIMENTS

Example systems, devices, methods, and computer-readable mediums are described herein. Any example, embodiment, or feature described herein is not necessarily to be construed as preferred or advantageous over other embodiments or features. The example embodiments described herein are not meant to be limiting. It will be readily understood that certain aspects of the disclosed devices, systems, and methods can be arranged and combined in a wide variety of different configurations, all of which are contemplated herein.

Furthermore, the particular arrangements shown in the figures should not be viewed as limiting. It should be understood that other embodiments might include more or less of each element shown in a given figure. Further, some of the illustrated elements may be combined or omitted. Yet further, an example embodiment may include elements that are not illustrated in the figures. Therefore, any component, feature, step or part may be integrated, separated, sub-divided, removed, duplicated, added, or used in any combination and remain within the scope of the present disclosure. Embodiments are exemplary only, and provide an illustrative combination of features, but are not limited thereto.

In an exemplary embodiment, a microdosing delivery system optimizes a dose of psychoactive drug and delivers the optimized dosage to a user based on one or more biological metrics.

In an exemplary embodiment of the present disclosure, a biological metric of a user is recorded and one or more quantifiable measures are derived from that signal. The measure is then compared to a target value or range, and dosage of a drug is adjusted in order to keep the measure close to a target or within a preset range.

In an exemplary embodiment, the biological metric may be received and analyzed for one or more quantifiable measures in order to set an initial dosage for a delivered drug. In an exemplary embodiment, the biological metric may be received and analyzed for one or more quantifiable measure in order to set a recurring dosage amount or interval for a delivered drug for a subsequent administration. In an exemplary embodiment, the system and method described herein may be used for the administration of a drug over time. The system and method may include a feedback loop to receive and analyze one or more biological metrics in order to determine one or more quantifiable measures to set an initial or recurring protocol of a delivered drug. The recurring protocol may be in a dosage amount, dosage strength, dosage duration, dosage interval, dosage drug, or combinations thereof.

The system and methods may include a dispensing system. The dispensing system may be used to make a determined dosage available to the user according to embodiments described herein, such as based on the determined one or more quantifiable measures to determine the drug, dosage amount, dosage strength, dosage duration, dosage interval, or combinations thereof. The dispensing system may be an automatic system to deliver the drug directly to the recipient's systems. The dispensing system may include a delivery component to provide the drug to the patient or caregiver to be administered to the patient. The dispensing system may include an indicator to provide notice, instructions, or other information to the patient or caregiver to administer the drug to the patient.

For example, an indicator may be provided to the user to indicate a time to ingest or a quantity of the drug to ingest. The indicator may be any signal and/or interface. The indicator may be a speaker to provide a sound, auditory instruction, or pattern of sounds/signals to indicate a quantity and/or duration of an administration of an associated drug. The indicator may be one or more lights to provide a similar indicator of a quantity and/or duration of an administration of an associated drug. The indicator may be a display screen to provide written and/or illustrated instructions for a quantity and/or duration of an administration of an associate drug. The indicator may be any combination or other interface to provide one or more visual, audial, or other sensory indication of a duration, amount, timing, or other administration parameter.

A user could have a quantity of pills and be instructed to take 1 pill every 10 minutes, and if the biological measure is low, the user is alerted to ingest 1 pill every 5 minutes, or it may alert the user to take 2 pills every 10 minutes. If the biological measure is high, the user is alerted to ingest 1 pill every 15 minutes. If the user is ingesting a liquid substance, the user could be alerted to drink 10 oz every 10 minutes, increase the amount to 12 oz every 10 minutes, or drink 10 oz every 8 minutes, depending on the relationship between the biological measure and the target value or range. Therefore, in general, the indicator may provide notice to the user to increase a dosage (through reducing a delay between dosings, increase an amount, increase concentration, or combinations thereof) if the biological measure is low and to decrease a dosage (through increasing a delay between dosings, decreasing an amount, or decreasing concentrations, or combinations thereof) if the biological measure is high.

In an exemplary embodiment of the present disclosure, the system could also automatically dispense an amount that the user should administer, such as through ingesting, inhaling, intravenous, dermal exposure, or other administration interface. Exemplary administration interfaces, therefore, may include, for example, a passage to deliver solid or liquid forms of the administered substance, a skin contact for application of a substance to the user's skin, a mask to provide administration of a gas to be inhaled by the user, or needle for subdermal and/or intravenous delivery of a substance to the user. Additionally, the device could dispense a measure of the substance, such as in solid or liquid form, (dispense liquid into a container or dispense a number of pills into a cup) with the expectation that the user will ingest the amount that is dispensed at the time it is dispensed.

In an exemplary embodiment of the present disclosure, adjustment of dosage is automatic where a calculated dosage of the drug is directly administered to the user. For example, an anesthetic may be administered via a catheter or marijuana smoke may be administered via a nasal cannula, and the dosage may be automatically adjusted based on the biological measure determined from the one or more biological signal. The dosage may be alerted by changing an amount of the substance delivered, by changing a concentration of the substance delivered, changing a duration of the quantity delivered, and combinations thereof.

The system and method may also include an input to determine, detect, and/or confirm the administration of the drug. Accordingly, for any of the dispensing systems used, or if a dispensing system is not use, the system may provide an input so the system receives dosage information about a dosage taken by the patient. The dosage information may include any combination of a starting dosage, a dosage concentration, a dosage amount, a dosage administration time when the dosage was actually provided to the patient's system (such as when the drug was ingested or injected or inhaled), a dosage delivery time when the dosage was provided to the patient, a dosage delivery form and/or method (such as pills for ingesting, liquid for ingesting, gaseous for inhalation, liquid for injection, etc.). The input may be any form, such as a button, toggle, touch screen, detector, sensor, or combinations thereof. For example, for a system that automatically administers and/or delivers a drug, the system may also record when the administration/delivery occurs. The system may also include a user interface, such as through a touch screen, keyboard, mouse, buttons, trackpad, etc. for receiving an input from a user that may be the patient or caregiver or other third party that may confirm or provide data to the system according to embodiments described herein.

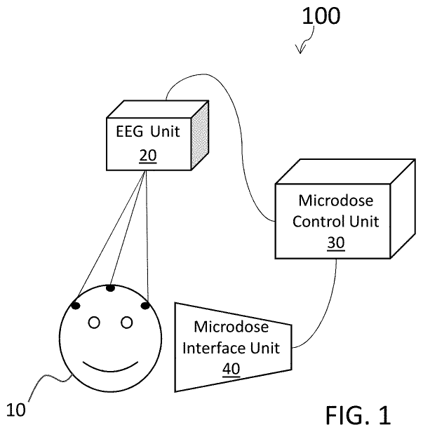

FIG. 1 depicts a microdosing system 100 according to an exemplary embodiment of the present disclosure.

The microdosing system 100 may be used by a user 10. The microdosing system 100 may include, but is not limited to, an EEG unit 20 communicatively-coupled to read electrical activity of the brain of the user 10, a microdose control unit 30 communicatively-coupled to the EEG unit 20 to receive biological metrics and control commands, and a microdose delivery interface 40 to interface with and deliver a microdose of a psychoactive drug to user 10.

Here, the user 10, can be any living creature with a biological metric according to embodiments described herein, such as an EEG or similar signal. The user 10 may be, but is not limited to, a human patient or other mammal. In an embodiment of the present disclosure, the user 10 will be undergoing psychoactive drug therapy to change the user's 10 mental state by affecting the brain and/or nervous system. Additionally, psychoactive drugs include, but are not limited to, LSD, peyote, psilocybin, delta-9 tetrahydrocannabinol (THC) and 3,4-methylenedioxy-methamphetamine (MDMA). Psychoactive drugs include any substance that may be used to affect the brain and/or nervous system.

The EEG unit 20 is any EEG machine capable of evaluating and monitoring the electrical activity of the user 10. In an embodiment of the present disclosure, the EEG unit 20 includes electrodes communicatively-coupled to the scalp of the user 10 to diagnostically detect changes in a predetermined EEG marker when a microdose of a psychoactive drug is administered to the user 10. The present disclosure and term EEG unit or EEG machine includes the full nineteen (19) recording electrodes of a conventional EEG. The EEG unit or EEG machine may also or alternatively refer to the use of more or less electrodes used to detect or monitor electrical activity of the user 10, such as for a subset of the electrodes or signals for different electrical activities within the user 10. The present disclosure includes but is not limited to an EEG for detecting or determining the one or more biological metric of the user 10. For example, other electrical signals from the brain may be received and analyzed similar to an EEG but that does not encompass the full nineteen (19) recording electrodes of a conventional EEG. The EEG unit or EEG machine may include one or more electrodes for detecting and/or determining an intrinsic frequency of a predefined EEG band, EEG alpha power of a predefined EEG band, Q-factor of a pre-defined intrinsic frequency of a predefined EEG band, coherence of an EEG across a plurality of locations on the brain, phase of an EEG across a plurality of locations on the brain, ratio of power of a first predefined EEG band and power of a second predefined EEG band, delta wave power, alpha2 power, alpha3 power, standard power measures, peak alpha frequency, EEG desynchronization, and/or global phase synchrony.

The microdose control unit 30 is communicatively-coupled to the EEG unit 20 to send and receive data about the user 10 and communicatively-coupled to the microdose delivery interface 40 to control microdose delivery of the psychoactive drug to the user 10.

In an embodiment of the present disclosure, the microdose control unit 30 includes, but is not limited to, at least one processer communicatively-coupled to at least one memory. The microdose control unit 30 receives data relating to the user 10 from the EEG unit 20 and determines whether to deliver, continue to deliver, increase delivery dosage, decrease delivery dosage, increase delivery rate/interval, decrease delivery rate/interval, and/or stop delivery of the psychoactive drug via the microdose delivery interface 40. The microdose control unit 30 may determine the delivery parameter of the administered substance. The microdose control unit 30 may include non-transitory machine readable code saved in memory that, when executed by the processor, is configured to perform the functions described herein, including determining the delivery parameter of the psychoactive drug via the microdose delivery interface and/or providing instructions to the microdose delivery interface to administer the psychoactive drug according to the determined delivery parameter.

In an embodiment of the present disclosure, the microdose control unit 30 is integrated with the microdose interface unit 40. As illustrated, the microdose control unit 30 is communicatively-coupled to the EEG unit 20 to send and receive data about the user 10 and communicatively-coupled to the microdose delivery interface 40 to control microdose delivery of the psychoactive drug to the user 10. However, each of the microdose control unit 30, the EEG unit 20, and/or the microdose delivery interface 40 may be integrated into one or more single housings and/or one or more separate housings. As illustrated, each resides in a separate housing that is separately and independently communicatively coupled to one or more other housing. In an exemplary embodiment, the microdose control unit 30 and the microdose interface unit 40 may be integrated into a single housing. In an exemplary embodiment, the EEG unit and the microdose interface unit may be integrated into a single housing. In an exemplary embodiment, the microdose control unit 30, microdose interface unit 40, and the EEG unit 20 are integrated into the same housing.

In an exemplary embodiment, one or more of the EEG unit 20 microdose control unit 30, and/or microdose interface unit 40 may be remote from other system components. For example, the EEG unit 20 may be a separate system that is administered to a patient either at the same location or another location. The microdose control unit 30 may then receive information and/or patient data from the EEG unit through a wired, wireless, combination of wired and wireless, or other communication connection. The microdose control unit 30 may thereafter analyze the EEG unit information and determine the desired dosage to administer of the substance to the patient.

The microdose delivery interface 40 is generally any electronically-controlled targeted drug delivery system capable of delivering a microdose of a drug to the user 10. This includes, but is not limited to, an electronic dispenser, an electronically-controlled pump, electronic transdermal patch, electronic nebulizer, patient-embedded microchip, electronic capsule and/or electronic pipette. Delivery methods include, but are not limited to, ingestion, injection, inhalation, and/or cutaneous contact, or other methods as described herein.

The microdose delivery interface 40 may be communicatively-coupled to the microdose control unit 30 via a hardwire connection and/or via a wireless connection to send and receive commands and data to/from the microdose control unit 30. The wireless connection can be, but is not limited to, a wireless personal area network (WPAN), wireless local area network (WLAN), wireless mesh network, cellular network, near-field communication (NFC) and/or Bluetooth.

In an embodiment of the present disclosure, the microdose delivery interface 40 is positioned adjacent to the user 10 and allows controlled delivery of a psychoactive drug to the user 10.

In an embodiment of the present disclosure, the microdose delivery interface 40 is directly interfaced with the user 10 and positioned either on the surface of the user 10, embedded within the user 10 and/or coupled to an intravenous (IV) line threaded through a vein of the user 10.

In another embodiment of the present disclosure, a storage container storing the psychoactive drug to be administered is integrated with the microdose delivery interface 40.

In yet another embodiment of the present disclosure, a storage container storing the psychoactive drug to be administered is separate from the microdose delivery unit 40 and is coupled to the microdose delivery interface 40 allowing transfer of the psychoactive drug to and from the storage container.

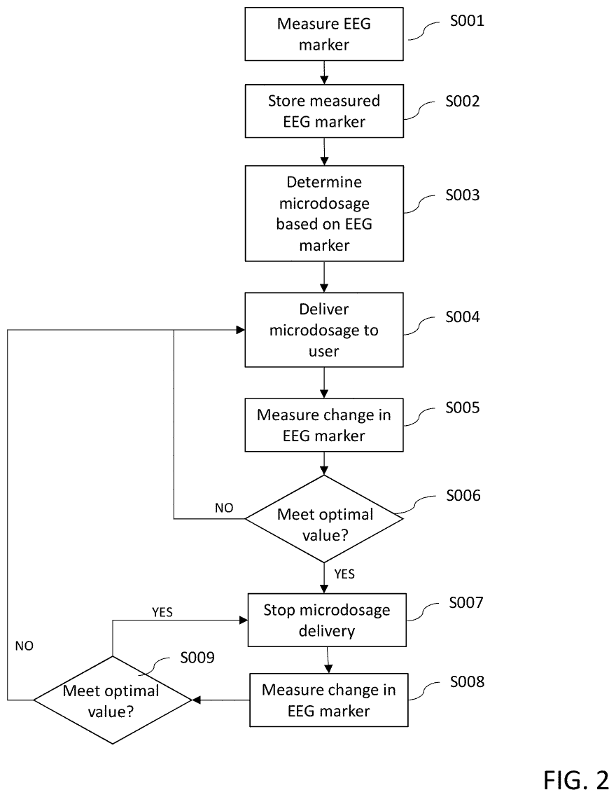

FIG. 2 is a flowchart according to an exemplary method embodiment of the present disclosure.

In step S001, before administering any psychoactive drug, the user 10 to receive microdosing is connected to a device to receive a biological metric(s) of the user 10, such as an EEG unit, and a predetermined baseline biological metric(s) or EEG marker is measured. The EEG marker can be, but is not limited to, an intrinsic frequency of a predefined EEG band, EEG alpha power of a predefined EEG band, Q-factor of a pre-defined intrinsic frequency of a predefined EEG band, coherence of an EEG across a plurality of locations on the brain, phase of an EEG across a plurality of locations on the brain, ratio of power of a first predefined EEG band and power of a second predefined EEG band, delta wave power, alpha2 power, alpha3 power, standard power measures, peak alpha frequency, EEG desynchronization, and/or global phase synchrony.

In an exemplary embodiment, the measured biological metric is a brain network that includes, but is not limited to, a salience network, a default mode network, an attention switching network, or combinations thereof.

In another exemplary embodiment, the measured biological metric may be a coherence, which includes transfer entropy for predetermined EEG bands with varying time epochs for processing. All, or any combination of the EEG markers described herein may be applied to specific EEG circuits such as the frontoparietal circuit. Exemplary embodiments may also include mutual information EEG markers, state transitions (between aforementioned networks), etc.

In an exemplary embodiment, the measured biological metric may be an event-related potentials such as p20, 50, 85, n100, p200, p300 amplitudes and latencies that may be tracked in response to dosage. Exemplary embodiments of the tracking of a biological metric may be used for repair/recovery/dosage monitoring entrainment or, EEG activity to a rhythmic visual strobe also for the same application of the visual strobe at the peak alpha frequency. Here, greater facilitation may be correspondent to an improvement in neuroplasticity as provided by appropriate dosage.

Exemplary embodiments may include composite scores or mulitvariates generated from all (or a portion) of the EEG markers described herein and may also be variables that may be measured and monitored.

Once the baseline biological metric measurement is obtained from the patient, the data is sent and/or received by the microdose control unit. The information may be received by the microdose control unit 30 in any manner. For example, the device for obtaining the baseline biological metric measurement may communicate the baseline biological metric to the microdose control unit. The microdose control unit 30 may retrieve the baseline biological metric through an intermediate data transfer, such as through communication over the internet, a network, or through a data storage device, such as a non-transitory machine readable memory drive.

In Step S002, the data is stored in a memory of the microdose control unit 30 to be used for determining a patient dosage delivery. Patient dosage delivery includes, but is not limited to, method of delivery, initial starting dosage, continuing dosage, dosage concentrations, dosage interval, and/or delivery duration.

At Step S003, a determination of microdosing parameters is made based on the baseline biological metric measurement. In an exemplary embodiment, the determination may be made through a comparison of the baseline EEG marker of the user and predetermined stored tables stored in the memory of the microdose control unit. In an exemplary embodiment, patient dosage is consistent at 1 unit per hour with nominal change in the biological metric measure of interest. Examples of the biological metric measurement include, but are not limited to, p300 amplitude, fronto-parietal cortical coherence, or wideband theta/alpha/beta activity. In the event that there is nominal change in the biological metric measure of interest, dosage is increased to 2 units per hour until biological measures of interest begin to change. At this point, dosage is maintained at 2 units per hour until there is a predefined threshold or predefined percentage change in the biological measure of interest that the measures fall within.

The system thereafter continues to monitor the biological measure of interest. If the values of the biological measure of interest move within a predetermined range, the dosage is reduced, and the variable is monitored. If the variable exits the predefined value threshold for the variable at any time, the dosage may be increased once again until the measure recovers back to the ascribed threshold. If the variable exits the predefined value threshold for the variable over a period of time, the dosage may be increased or decreased depending on the relative move of the variable outside of the predefined value threshold (for example if it goes up or down).

In another exemplary embodiment, dosage may be increased at specified time intervals until changes are noted in variables, or dosage may be increased only once per longer time period, where the longer time period corresponds with how fast-acting the psychoactive drug may be, how long it takes for the patient to metabolize the drug, or other factors associated with the drugs mechanism of action on the patient.

Additionally, dosing strategy may be varied where dosage may be reduced to 0 once a variable changes by a predetermined percentage or has an output variable move to a predetermined value range. This may follow a therapeutic schedule such as a sleep medication or drug that facilitates sleep, where the dosage is kept at a sufficient dosage to some variables which are not increased or decreased beyond some value range. This reduces side effects and/or facilitates deeper stages of sleep. Once determined, the microdosing parameters are communicated to the microdose delivery interface unit.

In Step S004, the delivery interface unit receives the microdosing parameters from the microdose control unit 30 and uses the information to dispense and/or administer a microdose of the psychoactive drug to the user 10. Additionally, the microdosing parameters include operating and control commands for the type of microdose delivery interface unit.

After administering/delivering the microdose, flow continues to Step S005 where a new measurement of the previous biological metric, such as an EEG marker, is measured by the device such as the EEG unit to determine the biological metric. The new measurement is then communicated to the microdose control unit 30 where a change in the biological metric (such as EEG marker value) from baseline is calculated based on the previously stored biological metric (such as EEG marker value).

Upon calculating the change in the baseline biological metric measurement (such as the EEG marker value), flow continues to Step S006 where the microdose control unit 30 determines whether the value falls within a predetermined optimal range to achieve an optimal outcome from the microdosing therapy.

In an embodiment of the present disclosure, the change in baseline biological metric measurement (such as EEG marker value) is compared to the lookup table previously stored and accessed in the memory of the microdose control unit.

In an embodiment of the present disclosure, the change in baseline biological metric measurement (such as EEG marker value) is compared to previously stored measurements.

If the change in the baseline biological metric measurement (EEG marker value) does not fall within an optimal range of change to achieve a predetermined optimal outcome from the microdosing therapy (NO in Step S006), the flow reverts back to Step S004. Here, an additional microdose is administered to the user 10 based on the previously determined microdosing parameters and flow continues to Step S005.

In an exemplary embodiment, a stimulant is microdosed and peak alpha frequency of a user 10 is monitored via an EEG where stimulant dosage is increased until an increase in peak alpha frequency is measured. When an increase is present, the increased stimulant dosage is maintained. If at the next predetermined time measurement, for example, 5 minutes later, peak alpha frequency is not faster than at the previous time point, stimulant dosage is increased once again. Alternatively, to maintain/reinforce the faster peak alpha frequency at the second time point, dosage may be maintained at the value at the second time point.

In another exemplary embodiment, the EEG may monitor activity at the left dorsolateral prefrontal cortex (LDPFC) of a user 10 and dosage may be stepwise or linear, with real-time monitoring of the activation of the LDPFC, which may be increases in beta and alpha frequency densities, as well as decreases in theta and delta activity. This may also correspond to changes in network coherence local to the region, with an increase in alpha coherence locally indicative of response to a dose, or non-response signaling for an increase in dose until these measures change as projected.

In yet another exemplary embodiment, the microdosing system can be used to determine and optimally reduce drug dosage. For example, dosage may be set at 100 units per hour and the EEG indicates activity or activations that indicate too much drug is being delivered to the user 10. This can be whether that metric be upregulation in delta and theta relative power density, coherence, mutual information, or connectivity measured in EEG electrodes globally (in front and posterior regions of the brain, both by themselves and as it relates to the same information between these regions). Thus, the microdose control unit 30 may be actively monitoring these variables, and provides an increase in dosage from a baseline zero or nonzero dosage. For example, if activation in pain circuitry, as measured via theta densities in dorsolateral prefrontal regions rise dramatically, the microdose interface unit may provide an increase in the drug until the increased theta density is reduced by some small or larger amount.

In a last exemplary embodiment, dosing may be facilitatory such as in a user 10 who suffers from a condition such as attention deficit disorder (ADD). Here, adaptive dosing is used to upregulate beta densities closely tied to attentional networks during academic lectures or, to facilitate focus at work/school.

The system may also determine based on the range of change, a modification to the dosage, such as duration, quantity, concentration, etc. The system may also or alternatively compare any combination of the current biological metric measurement, an amount or rate of change of the current biological metric measure from the immediately preceding or any preceding biological metric measures, or a baseline biological metric measurement to determine whether to proceed in Steps S006 (yes or no) to proceed to S004 or S007. For example, if the change between the current biological metric measurement and the immediately preceding biological metric measurement (or other predetermined preceding biological metric measurement) is below a first change threshold, and the current biological metric measurement is outside of a target range, above a target threshold, or above a target threshold, then, when the process proceeds to S004 to continue microdosing, the microdosing dosage may be altered. In this case, the system may determine that the patient is still outside a target condition for the biological metric, but that the change in the patient's condition with respect to the biological metric to reach the target condition is minimal. Therefore, the micro-dosing dosage may be increased. Alternatively, if the change between the current biological metric measurement and the immediately preceding biological metric measurement (or other predetermined preceding biological metric measurement) is above a second change threshold, and the current biological metric measurement is outside of a target range, above a target threshold, or above a target threshold, then, when the process proceeds to S004 to continue microdosing, the microdosing dosage may be altered. In this case, the system may determine that the patient is still outside a target condition for the biological metric, but that the change in the patient's condition with respect to the biological metric to reach the target condition is substantial or more than minimal. Therefore, the micro-dosing dosage may still be administered but may be at the same

If the change in baseline biological metric measurement (such as EEG marker value) does fall within an optimal range of change to achieve a predetermined optimal outcome from the microdosing therapy (YES in Step S006), the flow continues to Step S007. Here, microdosing is stopped and the user 10 is allowed to experience the optimal benefits of the microdosing therapy. In an exemplary embodiment the change in baseline biological metric measurement within an optimal range of change may be above or below a threshold value. The threshold value may correspond to the change and/or value of the biological metric measurement to indicate the user's response to the administered substance.

While the user 10 is experiencing benefits and dosage is stopped, flow periodically proceeds to S008, where a biological metric measurement (such as an EEG marker) is obtained. The change from baseline is evaluated in S009 to determine whether it is still within an optimal range of change. If it is (YES in Step S009), then the dosage continues to be stopped. Otherwise (NO in Step S009), flow proceeds to S004 for an additional dose delivery.

If over the course of administration of the drug there are notable changes in the EEG, the dosage rate may be altered accordingly. Specifically, in cases where minimal effective dosage (MED) may alter network function sufficient to expect downstream cognitive and functional differences, changes in wide or narrow-band coherence, network connectivity metrics, or time-dependent frequency architectures can be incorporated for monitoring.

In an exemplary embodiment, a user 10 may be administered a dosage of cannabidiol (CBD) at a rate of 0.01 ml/min with dosage concentration of 1 mg/ml and both posterior alpha and theta frequency architecture and connectivity is monitored. If resting network-posterior alpha coherence is negatively altered beyond a prespecified average threshold, or percentage threshold for baseline coherence, for example a set coherence value of 0.15, or percent change of 20% or greater, then the administered dosage may be considered to have had a network effect and dosage may be decreased.

Additionally, multiple frequency band measurements may be included such that there may be a drop in coherence. However, if wideband voltage of the alpha range (8-12 Hz) is incorporated, the subsequent dosage may only be reduced if, in addition to the previously noted drop in coherence, there is also a subsequent decrease in band density for a specific region, such as decrease in posterior alpha density beyond 1 uV, or 20% or greater.

The aforementioned changes may also be related to needs to increase medication if variance in these aforementioned or other metrics over the course of microdosing is close to 0. Variance of measures may also be considered as a vector indicative of dosage, where the average variance at values reaching over a preset standard deviation threshold, such as less than −1.0 or exceeding 1.0 is indicative of network response, and depending on the measure in question, subsequent action on dosage.

For example, a drop in average voltage or coherence of alpha band activity as compared to average activity for the individual, with average values less than −1.0 standard deviation from the mean for the individual or a population, may indicate initial network response and that dosage is sufficient and should be dropped to 0 after this has been realized for an hour. This value may be monitored after reducing dosage and dosage may once again be increased when average values are less skewed from the user's 10 baseline.

In another exemplary embodiment, multiple frequency band relationships may be incorporated algorithmically as part of the microdosing strategy. For example, a microdosing strategy may monitor activity in theta and alpha bands. In one application, bursts of alpha in their number, duration and occurrence as a baseline are monitored and scored, and a drug dose at 0.01 mg/min such that dosage is 1 mg/ml is administered (or desired rate and concentration based on the administered drug). Once both duration in terms of number of bursts per burst train, and occurrence of alpha bursts decreases, the dosage may be considered adequate for eliciting a response. However, in this case dosing may not be altered as this initial observation of alpha frequency architecture notes the beginning of cortical response to the drug.

As dosing continues, theta band activity is monitored, specifically for bursts of theta that develop as dosing continues indicative of further network change and response to the drug. Once theta bursting stabilizes or reaches a specific, present level, such as occurrences of 3 or more sequential wavelets in a burst in posterior regions, or amplitude of theta for narrow-band measurements is at 25% greater than the average pre-dosage amplitude, dosing is decreased or halted as the network has fully realized changes in response to the drug where additional administration may not further reinforce theta architecture or disrupt alpha architecture so as to keep overall dosage at a minimum to the user 10 while still maintaining network changes. Thus, a secondary benefit of the determination may be a signal to increase dosage.

In yet another exemplary embodiment, metrics for alpha network stability are disrupted or decreased, however theta metrics are not correspondingly increased. Here, dosage may be increased to 0.05 ml/min, or 3 mg/ml (or other corresponding rate and concentration based on the administered drug) and further administration of this drug may be provided until changes in theta architecture are realized. Then dosage is once again adjusted and decreased to a lower level or halted all together.

Measurements and determinations may include, but are not limited to, analysis for optimization and machine learning approaches, supervised and unsupervised approaches for populations or the user 10, and/or simulations for the user 10 such as random forest optimizations in an attempt to predict dosage course, response, and dynamically change or alter dosing strategy. Additionally, multivariates of measurements may be used to monitor for network response and correspondingly alter dosage and dosing plans.

Pursuant to these exemplary embodiments, a microdosing delivery system optimizes a dose of psychoactive drug and delivers the optimized dosage to a user 10 based on EEG determined biological metrics.

The functions of the above-described exemplary embodiments can also be realized by the following configuration. That is, the functions are also realized by supplying a program code for performing the processing of the above-described exemplary embodiments to a system or an apparatus and executing the program code by a computer (or a CPU or an MPU) of the system or the apparatus. In this case, the program code itself read from a storage medium realizes the functions of the above-described exemplary embodiments, and also the storage medium that stores the program code also realizes the functions of the above-described exemplary embodiments.

In addition, the configuration includes a case where the program code for realizing the functions of the above-described exemplary embodiments may be executed by a single computer (or a CPU or an MPU) and a case where the program code may be executed by a plurality of computers in cooperation with each other. Furthermore, the configuration includes a case where the program code may be executed by a computer, or hardware such as a circuit configured to realize the functions of the program code may be provided. As an alternative to the above, the configuration includes a case where part of the program code may be realized by hardware, and the remaining part may be realized by computer software.

Lastly, embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the invention is not limited to the disclosed exemplary embodiments. The scope of the following claims is to be accorded the broadest interpretation to encompass all modifications, equivalent structures and functions.

1. A microdosing delivery system configured to optimize a dose of psychoactive drug and deliver the optimized dosage to a user based on one or more biological metrics.

2. A microdosing delivery system of a drug, comprising:
a device for receiving a biological metric of a user;
a control unit for analyzing the biological metric to determine a dose of the drug; and
a dispensing system.

3. The microdosing delivery system of claim 2, wherein the control unit is configured to determine one or more quantifiable measures from the biological metric.

4. The microdosing delivery system of claim 3, wherein the control unit is configured to compare the one or more quantifiable measures to a target value or range, and determine a dosage of the drug in order to move or keep the one or more quantifiable measures close to or within the target or range.

5. The microdosing delivery system of claim 1, wherein the dispensing system is configured to make a determined dosage of the drug available to the user.

6. The microdosing delivery system of claim 5, wherein the dispensing system

7. The microdosing delivery system of claim 1, wherein the control system determines a dose of the drug by determining the drug, dosage amount, dosage strength, dosage duration, dosage interval, or combinations thereof.

8. The microdosing delivery system of claim 1, wherein the dispensing system is an automatic system configured to deliver the drug directly to the recipient's system.

9. The microdosing delivery system of claim 1, wherein the dispensing system is a delivery component to provide the drug to be administered to the patient.

10. The microdosing delivery system of claim 1, wherein the dispensing system is an indicator to provide notice, instructions, or other information to administer the drug to the patient.

11. The microdosing delivery system of claim 1, further comprising a system input configured to receive dosage information about a dosage taken by the patient.

12. The microdosing delivery system of claim 1, further comprising a storage container storing a psychoactive drug to be administered to the patient, the storage container separate from the microdose delivery unit and coupled to the microdose delivery interface allowing transfer of the psychoactive drug to and/or from the storage container.

13. A method of delivery a drug to a user, comprising:
receiving a biological metric from a patient;
determining a dosage of the drug based on the biological metric; and
administering the drug at the dosage to the patient.

14. The method of claim 13, further comprising: receiving a second biological metric from the patient after administering the drug at the dosage, and determining whether to change the dosage to a new dosage based on the second biological metric.

15. The method of claim 14, further comprising changing the dosage to the new dosage and administering the drug at the new dosage.

16. The method of claim 15, wherein the new dosage is at no dosage so that the drug delivery is stopped.

17. The method of claim 13, wherein the determining the dosage comprises comparing the biological metric or an attribute related to the biological metric is compared against a target value or target range.
